(19) United States
(12) Patent Application Publication
(10) Pub. No.: US 2022/0143484 A1
(43) Pub. Date: May 12, 2022

(54) SCORING SYSTEM OF AUTOMATICALLY DETECTING BODY MOTION, SCORING METHOD OF AUTOMATICALLY DETECTING BODY MOTION, AND NON-TRANSITORY COMPUTER-READABLE STORAGE MEDIUM

(71) Applicant: INSTITUTE FOR INFORMATION INDUSTRY, Taipei (TW)

(72) Inventors: Hsing-Man WANG, Taipei (TW); An-Chun CHEN, Taipei (TW); Yu-Chen CHENG, Taipei (TW)

(21) Appl. No.: 17/109,096

(22) Filed: Dec. 1, 2020

(30) Foreign Application Priority Data
Nov. 11, 2020 (TW) 109139401

(51) Int. Cl. A63B 71/06; A63B 24/00; G06N 20/00

(52) U.S. Cl. CPC A63B 71/0669; A63B 71/0622; A63B 24/0006; A63B 24/0062; A63B 2220/803; G06N 20/00; A63B 2071/0625; A63B 2024/0009; A63B 2024/0065; A63B 24/0075

(57) ABSTRACT

A scoring system of automatically detecting body motion includes a storage, a first motion sensor, and a processor. The storage is configured to store standard motion signal data corresponding to a multimedia signal, which the standard motion signal data includes a plurality of scoring segments, and the plurality of scoring segments are generated according to beat data of the multimedia signal and the standard motion signal data. The first motion sensor is configured to receive a first sensing signal, which the first sensing signal is generated by a set of user-motion according to the multimedia signal. The processor is configured to recognize user's motion signal of the first sensing signal and acquire to-be-scored-motion signal data corresponding to the plurality of scoring segments; and compare the to-be-scored-motion signal data corresponding to each of the plurality of scoring segments with the standard motion signal data to generate a score.

### CROSS-REFERENCE TO RELATED APPLICATION

This application claims priority to and the benefit of Taiwan Application Serial Number 109139401, filed on Nov. 11, 2020, the entire content of which is incorporated herein by reference as if fully set forth below in its entirety and for all applicable purposes.

### BACKGROUND

### Field of Disclosure

The disclosure generally relates to a scoring system and a scoring method, and more particularly to, a scoring system of automatically detecting motion and a scoring method of automatically detecting motion.

### Description of Related Art

As the sports market expands, users can go to the gym to attend the course to meet the instructor face to face, watch the course video or online live video to follow the virtual coach's motion or the instructor's motion to learn by the instructions and do the exercises. When the user watches the course video, the user can only do the exercise by following the motion of the instructor in the video alone and there is no way for the user to know whether his/her motion is accurate or what should be improved. Therefore, how to make the user know his/her motion is accurate when the user practices through the course video or distance learning is a technical problem urged to be improved.

### SUMMARY

The disclosure can be more fully understood by reading the following detailed description of the embodiments, with reference made to the accompanying drawings as described below. It should be noted that the features in the drawings are not necessarily to scale. In fact, the dimensions of the features may be arbitrarily increased or decreased for clarity of discussion.

One aspect of the present disclosure is to provide a scoring system of automatically detecting body motion including a storage, a first motion sensor, and a processor. The storage is configured to store standard motion signal data which corresponds to a multimedia signal, which the standard motion signal data includes a plurality of scoring segments, and the plurality of scoring segments are generated according to beat data of the multimedia signal and the standard motion signal data. The first motion sensor is configured to receive a first sensing signal, which the first sensing signal is generated by a set of user-motion according to the multimedia signal. The processor is communicatively coupled with the first motion sensor and the storage, and the processor is configured to: recognize a user's motion signal of the first sensing signal and acquire to-be-scored-motion signal data corresponding to the plurality of scoring segments; and compare the to-be-scored-motion signal data corresponding to each of the plurality of scoring segments with the standard motion signal data to generate a score.

One aspect of the present disclosure is to provide a scoring method of automatically detecting body motion including steps of storing standard motion signal data which corresponds to a multimedia signal, wherein the standard motion signal data comprises a plurality of scoring segments, the plurality of scoring segments are generated according to beat data of the multimedia signal and the standard motion signal data; receiving a first sensing signal by a first motion sensor, wherein the first sensing signal is generated by a set of user-motion according to the multimedia signal; recognizing user's motion signal of the first sensing signal and acquiring to-be-scored-motion signal data corresponding to the plurality of scoring segments; and comparing the to-be-scored-motion signal data corresponding to each of the plurality of scoring segments with the standard motion signal data to generate a score.

One aspect of the present disclosure is to provide a non-transitory computer-readable storage medium, including instructions stored thereon, the instructions being configured to cause a processor to store standard motion signal data which corresponds to a multimedia signal, wherein the standard motion signal data comprises a plurality of scoring segments, the plurality of scoring segments are generated according to beat data of the multimedia signal and the standard motion signal data; receiving a first sensing signal by a first motion sensor, wherein the first sensing signal is generated by a set of user-motion according to the multimedia signal; recognizing a user's motion signal of the first sensing signal and acquiring to-be-scored-motion signal data corresponding to the plurality of scoring segments; and comparing the to-be-scored-motion signal data corresponding to each of the plurality of scoring segments with the standard motion signal data to generate a score.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiments, with reference made to the accompanying drawings as described below. It should be noted that the features in the drawings are not necessarily to scale. In fact, the dimensions of the features may be arbitrarily increased or decreased for clarity of discussion.

FIG. 1 is a block diagram illustrating a scoring system of automatically detecting body motion according to some embodiments of the present disclosure.

FIG. 2 is a flow chart illustrating a scoring method of automatically detecting body motion according to some embodiments of the present disclosure.

FIG. 3 is a schematic diagram of standard motion signal data according to some embodiments of the present disclosure.

FIG. 4A to FIG. 4E is a schematic diagram of user's motion signal according to some embodiments of the present disclosure.

### DETAILED DESCRIPTION

The technical terms “first”, “second” and the similar terms are used to describe elements for distinguishing the same or similar elements or operations and are not intended to limit the technical elements and the order of the operations in the present disclosure. Furthermore, the element symbols/alphabets can be used repeatedly in each embodiment of the present disclosure. The same and similar technical terms can be represented by the same or similar symbols/alphabets in each embodiment. The repeated symbols/alphabets are provided for simplicity and clarity and they should not be interpreted to limit the relation of the technical terms among the embodiments.

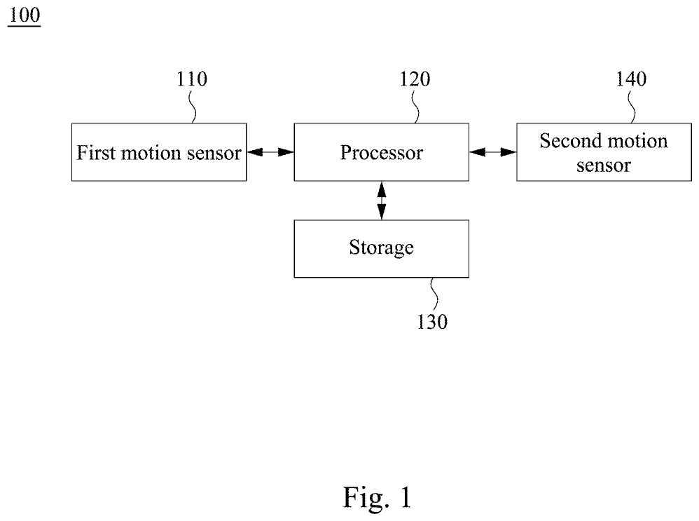

Reference is made to FIG. 1. FIG. 1 is a block diagram illustrating a scoring system 100 of automatically detecting body motion according to some embodiments of the present disclosure. As shown in FIG. 1, the scoring system 100 of automatically detecting body motion includes a first motion sensor 110, a processor 120, a storage 130, and a second motion sensor 140. The first motion sensor 110 is communicatively coupled with the processor 120. The processor 120 is communicatively coupled with the storage 130 and the second motion sensor 140.

In some embodiments, the first motion sensor 110 is worn by a user (e.g., a student) to acquire a signal when the user acts. The storage 130 stores standard motion signal data which corresponds to a multimedia signal. The standard motion signal data is the data that is collected by the second motion sensor 140 through the instructor's teaching motions. For example, the second motion sensor 140 is worn by the instructor (e.g., a coach), when the music or the video is played, the instructor acts the motions according to the beat of the music, and the second motion sensor 140 generates a sensing signal of the instructor's motions. The data is transmitted to the processor 120 for the following processing and is set to be the standard motion signal data of the music or video (i.e., the multimedia signal). Then, when the user listens to the same music and/or watches the same video and acts the indicated motions by following the same beat, the first motion sensor 110 generates the sensing signal while the user acts.

In some embodiments, the first motion sensor 110 receives the first sensing signal. The first sensing signal is generated by the set of user-motion according to the multimedia signal. The multimedia signal can be the music signal or the audio signal which includes the music signal. For example, the user watches the instructional video, in the meantime, the user listens to the music in the video and practices by imitating the instructor's motions.

In some embodiments, while constructing a scoring segment of a standard motion signal, the processor 120 computes the beat data of the music signal from the multimedia signal by using a machine learning algorithm. For example, the processor 120 applies the beats per minute (BPM) detection model as the machine learning algorithm. The BPM detection model computes the beat frequency of the inputted music to obtain a time length of each beat. The time length is applied as the beat data. It should be noted that one or more music songs may be played in a sports lesson or sports video and the music songs may include one or more beat patterns for different acts. Each music may have different beat patterns based on different music genres or rhythms. One music song may include one or more different beat patterns. The convention method of processing the BPM can only input changeless parameters, and the beat patterns may be changed in a music song and the starting point of the music may be not accurate, such problems result in the convention method of processing the BPM being not capable of acquiring the accurate beat pattern. In the present disclosure, the machine learning method is applied to resolve the problem of changeless parameters. For example, the convolution neural network (CNN) is applied to analyze the BPM of one or more music segments, and the corresponding beat data can be acquired from the multiple music segments of the multimedia signal, such that each scoring segment is determined accurately.

In some embodiments, the storage 130 stores the standard motion signal data which corresponds to the multimedia signal. The standard motion signal data is, for example, the instructors teaching motions, such as the angle data of the second sensing signal. Because the instructor listens to the music and acts the standard teaching motion at the same time, the motion of the instructor will correspond to the beat of the music.

In some embodiments, the standard motion signal data includes a plurality of scoring segments, and the scoring segments are generated according to the beat data of the multimedia signal and the standard motion signal data. For example, the beat data of the music signal is computed which is described above. Because the teaching motion of the instructor is generated according to the music signal, the second sensing signal generated by the second motion sensor 140 is the sensing signal which corresponds to the beat data in the timeline. In the sports exercise, the key action is considered to determine whether the posture is accurate to score a point. The key action presents a specific peak value in the standard motion signal data. In some embodiments, there may be multiple peak values of the standard motion signal data, and the peak values are not necessarily the same value. In other words, the processor 120 only has to set the beat data to be a tagging period according to the beat data computed from the music signal, finds the specific peak value (e.g., the peak value which is larger than a threshold) corresponding to the key action from the standard motion signal data, and then automatically tags the key action to create the scoring segment. There is no need to read the standard motion signal data of the instructor one-by-one manually to tag the key action. Therefore, the cost of manual operating can be reduced and the efficiency of creating the scoring segment is improved.

In some embodiments, the processor 120 recognizes user's motion signal of the first sensing signal. Then, the processor 120 acquires the to-be-scored-motion signal data corresponding to the plurality of scoring segments from the user's motion signal. For example, the user watches the motion of the instructor in the video, listens to the music in the video, and imitates the motion of the instructor to do the exercise. The first motion sensor 110 detects the user's motion to generate the first sensing signal, and the first sensing signal is transmitted to the processor 120. The processor 120 finds each corresponding scoring segment of the user's motion signal according to the scoring segment of the standard motion signal data of the instructor to obtain the to-be-scored-motion signal data.

In some embodiments, the processor 120 compares the to-be-scored-motion signal data corresponding to each of the plurality of scoring segments with the standard motion signal data to generate the score. For example, the processor 120 compares the to-be-scored-motion signal data with the standard motion signal data in each scoring segment. A determination of whether the user's motion is accurate can be made by comparing the motion signal data of the user with the motion signal data of the instructor. For example, if the to-be-scored-motion signal data satisfies or is similar to the standard motion signal data, it represents that the motion of the user is accurate. If the motion of the user is accurate, the score is increased. If the motion of the user is not accurate, the score is not added or is decreased, and the scoring rule is not limited herein. Then, the processor 120 generates the score for the user to refer to. Therefore, the user will know whether his/her motion is accurate by the score without the instructor aside.

In some embodiments, the first motion sensor 110 and the second motion sensor 140 can be the motion sensor, such as the inertial measurement unit (IMU). The first motion sensor 110 and the second motion sensor 140 are configured to detect the motion of the human body to generate and output the corresponding sensing signal, such as the angle signal, the acceleration signal, the angular velocity signal, the magnetic force signal, and the like, to be the first sensing signal and the second sensing signal. The motion signal which is to be scored can be one or the combination of the sensing signals described above.

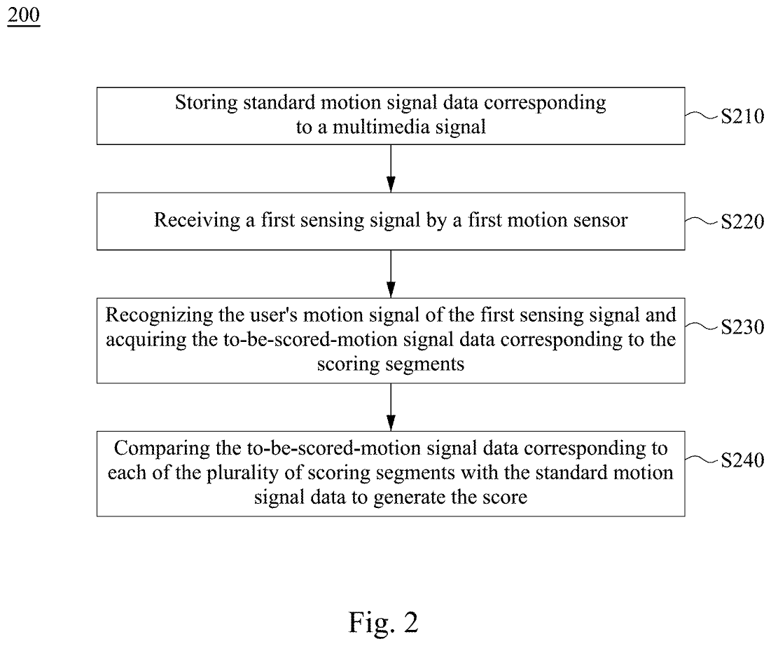

Reference is made to FIG. 2. FIG. 2 is a flow chart illustrating a scoring method 200 of automatically detecting body motion according to some embodiments of the present disclosure. The scoring system 100 of automatically detecting body motion in FIG. 1 can be used to execute the scoring method 200 of automatically detecting body motion. The description below is incorporated with FIG. 1 and FIG. 2.

In step S210, storing standard motion signal data corresponding to a multimedia signal is performed. In some embodiments, the standard motion signal data is obtained by recognizing the sensing signal of the instructor according to the beat of the music.

In some embodiments, the standard motion signal data includes a plurality of scoring segments, and the plurality of scoring segments are generated by the beat data of the multimedia signal and the standard motion signal data. For example, the time section that the instructor acts corresponds to the beat of the music signal, such that the motion signal of the instructor corresponds to the beat of the music. The motion signals which correspond to the beat of the music are set to be the standard motion signal data.

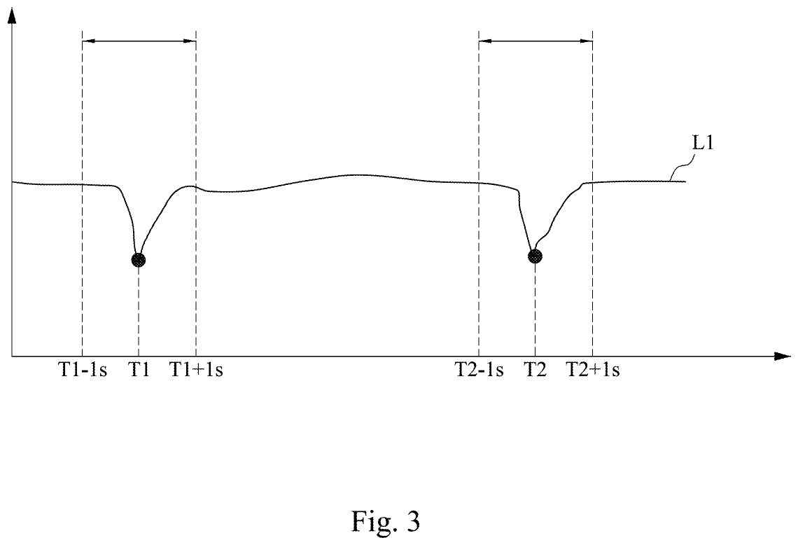

In some embodiments, the motion signal which will be scored is, for example, the angle signal. Reference is made to FIG. 3. FIG. 3 is a schematic diagram of standard motion signal data L1 which represents the wave variance of the angle signal in the timeline according to some embodiments of the present disclosure. The instructor does the action, such as a punch or a kick, by following the beat of the music. When the instructor does the action, the angle signal has a larger variance and the peak value.

Reference is further made to FIG. 1 and FIG. 2. In step S220, receiving the first sensing signal by the first motion sensor 110 is performed. In some embodiments, the first sensing signal is generated by the set of user-motion according to the multimedia signal. For example, the user watches the video of the instructor and imitates the motion of the instructor by the music rhythm for learning.

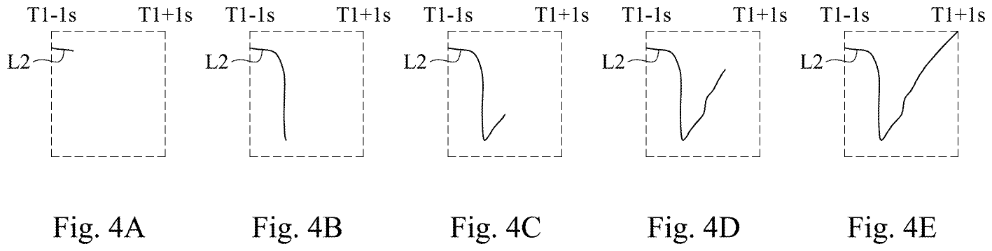

Reference is further made to FIG. 2. In step S230, recognizing the user's motion signal of the first sensing signal and acquiring the to-be-scored-motion signal data corresponding to the scoring segments is performed. In some embodiments, the to-be-scored-motion signal data is read at multiple time points of the scoring segment. FIG. 4A to FIG. 4E are schematic diagrams of the user's motion signal L2 of the scoring segment T1 according to some embodiments of the present disclosure. The embodiment shows the to-be-scored-motion signal data which is read at the five time points respectively. It should be noted that the reading-times number is not limited herein.

Reference is further made to FIG. 2. In step S240, comparing the to-be-scored-motion signal data corresponding to each of the plurality of scoring segments with the standard motion signal data to generate the score is performed. In FIG. 3, FIG. 4A to FIG. 4E, when a determination of whether the user's motion is accurate is made, a key action scoring segment which is computed from the second motion sensor and the multimedia signal is applied to take the user's motion signal of the first motion sensor as the to-be-scored-motion signal data. In step S240, the to-be-scored-motion signal data in FIG. 4A to FIG. 4E is compared with the standard motion signal data to obtain the five determination results. The determination results are computed to generate the score.

In some embodiments, the scoring method 200 of automatically detecting body motion computes the beat of the audio signal of the multimedia signal by using the machine learning algorithm to be the beat data. For example, the beats per second (BPM) of the music is trained and detected by using the convolutional neural network (CNN).

In some embodiments, the scoring method 200 of automatically detecting body motion sets the tagging period according to the time length corresponding to the beat data of the audio signal and reads a plurality of peak values from the standard motion signal data according to the tagging period to determine the scoring segment. For example, the time length is computed from the beat data of the audio signal, such as the time length between each beat. The time length is used as the tagging period for reading the peak value in the standard motion signal data. When the time length between the peak value A and the other peak value which is in front of the peak value A is larger than the tagging period, a determination that the peak value A corresponds to a key action signal B is made and the scoring segment is determined according to the position of the peak value A, such as 1 second before and after the peak value A. The scoring method 200 of automatically detecting body motion can tag the key action of the standard motion signal data according to the tagging period which is computed by the beat data of the music signal and the peak value of the standard motion signal data.

In some embodiments, the scoring method 200 of automatically detecting body motion uses the angle data of the first sensing signal corresponding to the user as the to-be-scored-motion signal data according to each scoring segment. Taking that the length of the scoring segment is 2 seconds as an example. The scoring method 200 of automatically detecting body motion takes the time T1 as a base point, and the time section between 1 second before and after the time T1 is applied to execute a comparison of the sensing signal (e.g., the time segment from the T1−1 second to the T1+1 second shown in FIG. 3 and FIG. 4A to FIG. 4E), and the sampling process for the comparison is executed multiple times in the time section. For example, FIG. 4A to FIG. 4E shows five-times sampling for the comparison of the to-be-scored-motion signal data in the comparison section. The scoring method 200 of automatically detecting body motion compares the to-be-scored-motion signal data with the standard motion signal data multiple times, such that the sensing signals of the user can be compared multiple times. If one action of the user is early or late, the determination that the complete state or the similarity of the motion in the scoring segment can be still made by multiple comparisons.

In some embodiments, the multimedia signal includes different beats based on the design. For example, the beat data of the multimedia signal includes many beats having different time lengths. For the sake of brevity, two different time lengths of the beats are shown as an embodiment and described below.

In some embodiments, the multimedia signal includes multiple beats. The beat data includes a first beat and a second beat. As described above, the scoring method 200 of automatically detecting body motion computes the first beat and the second beat of the audio signal of the multimedia signal by using the machine learning algorithm. Then, the time length of the first beat is set to be a first tagging period of the multimedia signal and the time length of the second beat is set to be a second tagging period of the multimedia signal.

In some embodiments, the scoring method 200 of automatically detecting body motion computes the first scoring segment and the second scoring segment by using the first tagging period and the second tagging period. For example, the instructor may change music which has different beats or a piece of music has different beats in some sports video. The instructor does the motion by following the different beats (e.g., the punch or the kick). In the step of determining the scoring segment according to the beat data of the audio signal, the scoring method 200 of automatically detecting body motion generates the first tagging period according to the first beat data, reads the plurality of the peak values of the sensing signal of the instructor, and records the first scoring segment which is the time section between the peak values (satisfying the length of the first tagging period). Similarly, when the music is changed to be the second beat, the second tagging period is generated according to the second beat data, the plurality of peak values of the sensing signal of the instructor, and the second scoring segment which is the time section between the peak values is recorded (satisfying the length of the second tagging period).

In some embodiments, the scoring method 200 of automatically detecting body motion determines a sampling window by each corresponding scoring segment, which the length of the sampling window is smaller than the length of the scoring segment. In the sampling window, the to-be-scored-motion signal data of the user is compared with the standard motion signal data of the instructor to compute the score. For example, the time length of the scoring segment is 2 seconds. The scoring method 200 of automatically detecting body motion compares the to-be-scored-motion signal data of the user with the standard motion signal data of the instructor six times per second (i.e., multiple sampling windows), for example. Then, the value which has the largest similarity between the to-be-scored-motion signal data of the user and the standard motion signal data of the instructor is outputted to be the comparison result.

In some embodiments, the scoring method 200 of automatically detecting body motion generates feedback information corresponding to the score to provide the user as a consulting report, such that the user knows whether his/her motion is accurate and how much difference between the instructor's motion and his/her motion.

In some circumstances, there may be a discrepancy between the time sequence of the first sensing signal of the user and the time sequence of the multimedia signal. When the discrepancy in time series exists, the comparison will be not accurate. In some embodiments, the scoring method 200 of automatically detecting body motion executes the timing correction algorithm to calibrate the time according to the time tags of the first sensing signal and the multimedia signal to align the time sequences of the first sensing signal and the multimedia signal.

In some embodiments provides a non-transitory computer-readable storage medium storing multiple instructions. When the instructions are loaded into the processor or the processor 120 in FIG. 1, the processor 120 executes the instructions to perform steps of FIG. 2. For example, the processor 120 stores the standard motion signal data corresponding to the multimedia signal and receives the first sensing signal by the first motion sensor 110. Then, the processor 120 recognizes the user's motion signal of the first sensing signal and acquires the to-be-scored-motion signal data corresponding to the scoring segments. The processor 120 compares the to-be-scored-motion signal data corresponding to each scoring segment with the standard motion signal data to generate the score.

Accordingly, the scoring system of automatically detecting body motion and the scoring method of automatically detecting body motion in the present disclosure provides the user to watch synchronous/asynchronous and online/offline videos. The sensing information provided by the sensor which is worn on the user can be sent to the system to determine whether the user's motion is accurate. Furthermore, there is no need to tag the motion signal of the instructor by manual work. Instead, the key action of the instructor is determined automatically by the beat data of the music and tagged automatically. Not only the time cost and the manual cost for tagging the key action of the instructor is decreased, but also the mistake of manual tagging is avoided. For example, when manual tagging is performed, the larger signal value such as the wave peak is tagged as the key action. The continuous actions affect the signal wave, such that the signal value is larger than the minimum value but smaller than the actual maximum value and the manual tagging is still made because of the erroneous determination. In the present disclosure, the method of automatically tagging the key action of the instructor can prevent from tagging the key action manually, and the method of automatically tagging the key action of the instructor can prevent the problems.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A scoring system of automatically detecting body motion, comprising:
a storage configured to store standard motion signal data which corresponds to a multimedia signal, wherein the standard motion signal data comprises a plurality of scoring segment and the plurality of scoring segments are generated according to beat data of the multimedia signal and the standard motion signal data;
a first motion sensor configured to receive a first sensing signal, wherein the first sensing signal is generated by a set of user-motion according to the multimedia signal; and
a processor communicatively coupled with the first motion sensor and the storage, wherein the processor is configured to:
recognize a user's motion signal of the first sensing signal and acquire to-be-scored-motion signal data corresponding to the plurality of scoring segments; and
compare the to-be-scored-motion signal data corresponding to each of the plurality of scoring segments with the standard motion signal data to generate a score.

2. The scoring system of automatically detecting body motion of claim 1, wherein the processor is further configured to:
compute a beat of an audio signal of the multimedia signal to be the beat data by using a machine learning algorithm;
set a tagging period by a time length corresponding to the beat data of the audio signal; and
read a plurality of peak values from the standard motion signal data according to the tagging period and determine the plurality of scoring segments.

3. The scoring system of automatically detecting body motion of claim 2, further comprising:
a second motion sensor coupled to the processor, wherein the second motion sensor is configured to receive a second sensing signal, wherein the second sensing signal is generated by a set of instructor-motion according to the multimedia signal.

4. The scoring system of automatically detecting body motion of claim 3, wherein the standard motion signal data comprises angle data.

5. The scoring system of automatically detecting body motion of claim 4, wherein the processor is further configured to:
set the to-be-scored-motion signal data by the angle data of the first sensing signal corresponding to the user for each scoring segment and compare the to-be-scored-motion signal data corresponding to each scoring segment with the standard motion signal data, that is, compare the angle data of the user with the angle data of the instructor in each scoring segment to determine whether the angle data of the user-motion and the angle data of the instructor-motion is similar.

6. The scoring system of automatically detecting body motion of claim 2, wherein in different sections corresponding to the multimedia signal, the beat data comprises a first beat and a second beat, the tagging period comprises a first tagging period and a second tagging period, and the processor is further configured to:
compute the first beat and the second beat of the audio signal of the multimedia signal by using the machine learning algorithm; and
set the first tagging period of the multimedia signal by the time length corresponding to the first beat and set the second tagging period of the multimedia signal by the time length corresponding to the second beat.

7. The scoring system of automatically detecting body motion of claim 6, further comprising:
a second motion sensor coupled to the processor, wherein the second motion sensor is configured to receive a second sensing signal, and the second sensing signal is generated by a set of instructor-motion according to the multimedia signal;
wherein the processor is further configured to read a peak value of the second sensing signal in the first tagging period to record the section corresponding to the peak value to be the scoring segment in the first tagging period; and
read the peak value of the second sensing signal in the second tagging period to record the section corresponding to the peak value to be the scoring segment in the second tagging period.

8. The scoring system of automatically detecting body motion of claim 1, wherein the multimedia signal corresponding to each scoring segment comprises a time tag, and the processor is further configured to execute a timing correction algorithm to correct a time sequence of the first sensing signal and the multimedia signal according to the first sensing signal and the time tag of the multimedia signal.

9. The scoring system of automatically detecting body motion of claim 1, wherein the processor is further configured to:
determine a sampling window from each scoring segment and compare the to-be-scored-motion signal data of the user with the standard motion signal data in the sampling window to compute the score.

10. The scoring system of automatically detecting body motion of claim 1, wherein the processor is further configured to:
generate feedback information corresponding to the score.

11. A scoring method of automatically detecting body motion, comprising:
storing standard motion signal data which corresponds to a multimedia signal, wherein the standard motion signal data comprises a plurality of scoring segments, the plurality of scoring segments are generated according to beat data of the multimedia signal and the standard motion signal data;
receiving a first sensing signal by a first motion sensor, wherein the first sensing signal is generated by a set of user-motion according to the multimedia signal;
recognizing a user's motion signal of the first sensing signal and acquiring to-be-scored-motion signal data corresponding to the plurality of scoring segments; and
comparing the to-be-scored-motion signal data corresponding to each of the plurality of scoring segments with the standard motion signal data to generate a score.

12. The scoring method of automatically detecting body motion of claim 11, further comprising:
computing a beat of an audio signal of the multimedia signal to be the beat data by using a machine learning algorithm;
setting a tagging period by a time length corresponding to the beat data of the audio signal; and
reading a plurality of peak values from the standard motion signal data according to the tagging period and determining the plurality of scoring segments.

13. The scoring method of automatically detecting body motion of claim 12, further comprising:
receiving a second sensing signal by a second motion sensor, wherein the second sensing signal is generated by a set of instructor-motion according to the multimedia signal.

14. The scoring method of automatically detecting body motion of claim 13, wherein the standard motion signal data comprises angle data.

15. The scoring method of automatically detecting body motion of claim 14, further comprising:
setting the to-be-scored-motion signal data by the angle data of the first sensing signal corresponding to the user for each scoring segment and comparing the to-be-scored-motion signal data corresponding to each scoring segment with the standard motion signal data, that is, comparing the angle data of the user with the angle data of the instructor in each scoring segment to determine whether the angle data of the user-motion and the angle data of the instructor-motion is similar.

16. The scoring method of automatically detecting body motion of claim 12, wherein in different sections corresponding to the multimedia signal, the beat data comprises a first beat and a second beat, the tagging period comprises a first tagging period and a second tagging period, and the scoring method of automatically detecting body motion further comprises:
computing the first beat and the second beat of the audio signal of the multimedia signal by using the machine learning algorithm; and
setting the first tagging period of the multimedia signal by the time length corresponding to the first beat and setting the second tagging period of the multimedia signal by the time length corresponding to the second beat.

17. The scoring method of automatically detecting body motion of claim 16, further comprising:
receiving a second sensing signal by a second motion sensor, wherein the second sensing signal is generated by a set of instructor-motion according to the multimedia signal;
reading a peak value of the second sensing signal in the first tagging period to record the section corresponding to the peak value to be the scoring segment in the first tagging period; and
reading the peak value of the second sensing signal in the second tagging period to record the section corresponding to the peak value to be the scoring segment in the second tagging period.

18. The scoring method of automatically detecting body motion of claim 11, wherein the multimedia signal corresponding to each scoring segment comprises a time tag, and the scoring method further comprises:
executing a timing correction algorithm to correct a time sequence of the first sensing signal and the multimedia signal according to the first sensing signal and the time tag of the multimedia signal.

19. The scoring method of automatically detecting body motion of claim 11, further comprising:
determining a sampling window from each scoring segment and comparing the to-be-scored-motion signal data of the user with the standard motion signal data in the sampling window to compute the score.

20. The scoring method of automatically detecting body motion of claim 11, further comprising:
generating feedback information corresponding to the score.

21. A non-transitory computer-readable storage medium, comprising instructions stored thereon, the instructions being configured to cause a processor to:
storing standard motion signal data which corresponds to a multimedia signal, wherein the standard motion signal data comprises a plurality of scoring segments, the plurality of scoring segments are generated according to beat data of the multimedia signal and the standard motion signal data;
receiving a first sensing signal by a first motion sensor, wherein the first sensing signal is generated by a set of user-motion according to the multimedia signal;
recognizing a user's motion signal of the first sensing signal and acquiring to-be-scored-motion signal data corresponding to the plurality of scoring segments; and
comparing the to-be-scored-motion signal data corresponding to each of the plurality of scoring segments with the standard motion signal data to generate a score.
